(19) United States
(12) Patent Application Publication
(10) Pub. No.: US 2005/0106242 A1
(43) Pub. Date: May 19, 2005

(54) Melt blend dispersions

(76) Inventors: Dong Yan, Cupertino, CA (US); Liang Dong, Sunnyvale, CA (US); Patrick Wong, Burlingame, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/987,237

(22) Filed: Nov. 12, 2004

Related U.S. Application Data
(60) Provisional application No. 60/519,581, filed on Nov. 13, 2003.

(51) Int. Cl. A61K 31/56; A61K 31/55; A61K 9/48; A61K 31/19; A61K 9/20

(52) U.S. Cl. 424/464; 514/171; 514/217; 514/254.07; 514/570

(57) ABSTRACT

The present invention relates to compositions and methods for enhancing the dissolution and bioavailibilty of beneficial agents with low water solubility.

### CROSS-REFERENCE TO RELATED APPLICATIONS

This application claims benefit of U.S. provisional patent application No. 60/519,581, filed Nov. 13, 2003, which is incorporated herein by reference.

### FIELD OF THE INVENTION

The present invention relates to compositions and methods for enhancing the dissolution and bioavailibilty of beneficial agents with low water solubility.

### BACKGROUND OF THE INVENTION

Enhancing the dissolution and bioavailibilty of beneficial agents with low water solubility is of great interest in the art. Such compounds include all those that can be categorized as Class 2 by the United States Food and Drug Administration (FDA), which has issued a set of guidelines outlining the Biopharmaceutical Classification System (BCS). The BCS is a scientific framework for classifying drug substances based on their aqueous solubility and intestinal permeability. When combined with the dissolution of the drug product, the BCS takes into account three major factors that govern the rate and extent of drug absorption from IR solid oral dosage forms: dissolution, solubility, and intestinal permeability. According to the BCS, drug substances are classified as follows: Class 1: High Solubility—High Permeability; Class 2: Low Solubility—High Permeability; Class 3: High Solubility—Low Permeability; and Class 4: Low Solubility—Low Permeability. With Class 2 drugs, dissolution/solubilization in the gastro-intestinal tract and luminal transport of the dissolved molecules is the limiting step for absorption, and thus increasing dissolution rates is an important goal.

Dissolution rates may be increased by numerous approaches, including reducing the particle size of the beneficial agent to the order of a few microns or less to increase the surface area.

In the past, mechanical approaches such as ball milling, air jet milling, and high-pressure homogenization have been used to decrease particle size. However, these methods are limited, in that the crystalline structure of the beneficial agent remains unchanged. Moreover, the processes are time consuming and require a great deal of energy for a comparatively low yield.

In contrast, the present invention comprises compositions and methods involving solid dispersions where the beneficial agent particles are homogeneously distributed throughout a solid matrix carrier. Solid dispersions provide the capability to decrease the particle size of a beneficial agent to a nearly molecular level, lower the melting point of the beneficial agent, and reduce the crystallinity of the beneficial agent, thereby enhancing the dissolution and bioavailibilty of beneficial agents with low water solubility.

### SUMMARY OF THE INVENTION

The present invention describes methods of preparing solid dispersions for delivering beneficial agents with low water solubility, comprising melting the beneficial agents with polymeric carriers, homogenizing the resulting mixtures, and cooling the mixtures.

Methods of preparing solid dispersions for delivering beneficial agents with low water solubility to patients are described, the methods comprising melting the beneficial agents with polymeric carriers, homogenizing the resulting mixtures, cooling the mixtures, and administering the dispersed beneficial agents to patients.

### BRIEF DESCRIPTION OF THE DRAWINGS

FIG. 1 is a graph of frequency percentage versus particle size for pure progesterone.

FIG. 2 is a graph of frequency percentage versus particle size for a composition of the present invention.

FIG. 3 is a graph of DSC curves for pure progesterone, compositions of the present invention, and a self emulsifying formulation.

FIG. 4 is a graph showing dissolution profiles for pure progesterone, compositions of the present invention, and a self emulsifying formulation.

FIG. 5 is a graph of the particle size of precipitated progesterone from compositions of the present invention versus the viscosity of polymeric carrier.

FIG. 6 is a graph of the percentage of drug released over time for a composition of the present invention.

### DETAILED DESCRIPTION

The present invention describes methods of preparing solid dispersions for delivering beneficial agents with low water solubility, comprising melting the beneficial agents with polymeric carriers, homogenizing the resulting mixtures, and cooling the mixtures.

Melting the beneficial agent with the polymeric carrier is achieved by heating the mixture to a temperature greater than the melting point of the polymeric carrier, but lower than the temperature where the beneficial agent or polymeric carrier will degrade. Most carriers melt in a range from about −40° C. to about 60° C. No harm comes from heating the beneficial agent and carrier mixture to higher temperatures, provided that the temperature is lower than the temperature where the beneficial agent or polymeric carrier will degrade. Also, the beneficial agent need not be melted, as it will still solubilize in the melted carrier. Preferably, the mixture is heated in a range from about 60° C. to about 150° C. More preferably to about 135° C.

Homogenizing is achieved using conventional methods.

Cooling is achieved using conventional methods. Preferably, the mixture is rapidly cooled.

Beneficial agents used in the present invention include all those compounds known to have an effect on humans or animals that also have low water solubility. Such compounds include all those that can be categorized as Class 2 under the Biopharmaceutical Classification System (BCS) set out by the United States Food and Drug Administration (FDA). Determining which BCS Class a drug bellows in is a matter of routine experimentation, well known to those skilled in the art.

Exemplary beneficial agents that can be delivered by the osmotic system of this invention include prochlorperazine edisylate, ferrous sulfate, aminocaproic acid, potassium chloride, mecamylamine hydrochloride, procainamide hydrochloride, amphetamine sulfate, benzphetamine hydrochloride, isoproternol sulfate, methamphetamine hydrochloride, phenmetrazine hydrochloride, bethanechol chloride, metacholine chloride, pilocarpine hydrochloride, atropine sulfate, methascopolamine bromide, isopropamide iodide, tridihexethyl chloride, phenformin hydrochloride, methylphenidate hydrochloride, oxprenolol hydrochloride, metroprolol tartrate, cimetidine hydrochloride, diphenidol, meclizine hydrochloride, prochlorperazine maleate, phenoxybenzamine, thiethylperazine, maleate, anisindone, diphenadione erythrityl teranitrate, digoxin, isofurophate, reserpine, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 β-estradiol, ethinyl estradiol, ethinyl estradiol 3-methyl ether, prednisolone, 17-hydroxyprogesterone acetate, 19-nor-progesterone, norgestrel orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of a-methyldopa hydrochloride, theophylline, calcium gluconate ferrous lactate, ketoprofen, ibuprofen, cephalexin, haloperiodol, zomepirac, vincamine, diazepam, phenoxybenzamine, nifedipine, diltiazen, verapamil, lisinopril, captopril, ramipril, fosimopril, benazepril, libenzapril, cilazapril cilazaprilat, perindopril, zofenopril, enalapril, indalapril, qumapril, and the like. Other beneficial agents are known to the dispensing art as described in Pharmaceutical Sciences, by Remington, 14th Ed., 1979, published by Mack Publishing Co., Easton, Pa.; The Beneficial agent, The Nurse, The Patient, Including Current Beneficial agent Handbook, 1976, by Falconer et al., published by Saunder Company, Philadelphia, Pa.; Medical Chemistry, 3rd Ed., Vol. 1 and 2, by Burger, published by Wiley-Interscience, New York; and, Physician's Desk Reference, 55nd Ed., 1998, published by Medical Economics Co., New Jersey. The beneficial agent may be in various forms such as unchanged molecules, molecular complexes, pharmacologically acceptable salts such as hydrochloride, hydrobromide, sulfate, laurate, palmitate, phosphate, nitrite, nitrate, borate, acetate, maleate, tartrate, oleate, salicylate, and the like. For acidic beneficial agents, salts of metals, amines, or organic cations, for example quaternary ammonium can be used. Derivatives of beneficial agents, such as bases, ester, ether and amide can be used.

Beneficial agents having low water solubility, e.g., less than 50 micrograms/ml, are useful with the present invention. Beneficial agents include progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the beneficial agents include progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include progesterone, megestrol acetate, topiramate, and naproxen. Most preferably, the beneficial agent is progesterone.

In one embodiment of the method, the beneficial agent is present in a range from about 0.0001 percent to about 95 percent by weight of the composition. Preferably, the beneficial agent is present in a range from about 1 percent to about 20 percent by weight of the composition.

In one embodiment of the method, the carrier to beneficial agent ratio is about 10 to about 1.

In one embodiment of the method, the carrier to beneficial agent ratio is about 10 to about 5.

The carrier is a block copolymer of propylene oxide and ethylene oxide, a block copolymer derived from the addition of ethylene oxide and propylene oxide to ethylenediamine, polyethelene glycol, or polyethylene oxide.

In one embodiment of the method, the block copolymer of propylene oxide and ethylene oxide is of a formula HO-(ethylene oxide)ₓ-(propylene oxide)_{y}-(ethylene oxide)_{x′}-H. Preferably, x is in a range from about 2 to about 150, y is in a range from about 20 to about 70, and x′ is in a range from about 2 to about 150. Block copolymers of propylene oxide and ethylene oxide are available under the tradename PLURONIC from BASF Corporation, New Jersey, USA. PLURONIC block copolymers are pharmaceutical excipients listed in the US and British Pharmacopoeia.

Where relatively lower beneficial agent concentrations are desired, i.e., where the carrier to beneficial agent ratio is about 10 to about 1, preferably, x is in a range from about 20 to about 150, y is in a range from about 20 to about 70, and x′ is in a range from about 20 to about 150.

Where relatively higher beneficial agent concentrations are desired, i.e., where the carrier to beneficial agent ratio is about 10 to about 5, preferably, x is in a range from about 2 to about 80, y is in a range from about 20 to about 70, and x′ is in a range from about 2 to about 80.

In one embodiment of the method, x is about 41, y is about 16, and x′ is about 41. Such a block copolymer of propylene oxide and ethylene oxide is available under the tradename PLURONIC F38.

In one embodiment of the method, x is about 79, y is about 28, and x′ is about 79. Such a block copolymer of propylene oxide and ethylene oxide is available under the tradename PLURONIC F68.

In one embodiment of the method, x is about 64, y is about 37, and x′ is about 64. Such a block copolymer of propylene oxide and ethylene oxide is available under the tradename PLURONIC F87.

In one embodiment of the method, x is about 26, y is about 39, and x′ is about 26. Such a block copolymer of propylene oxide and ethylene oxide is available under the tradename PLURONIC P85.

In one embodiment of the method, x is about 141, y is about 44, and x′ is about 141. Such a block copolymer of propylene oxide and ethylene oxide is available under the tradename PLURONIC F108.

In one embodiment of the method, x is about 101, y is about 56, and x′ is about 101. Such a block copolymer of propylene oxide and ethylene oxide is available under the tradename PLURONIC F127.

Block copolymers derived from the addition of ethylene oxide and propylene oxide to ethylenediamine are available under the tradename TETRONIC from BASF Corporation, New Jersey, USA. The hydrophobic and hydrophilic parts of the molecule can be selectively varied to give a wide range of functional characteristics to give specific carrier requirements, and selection of the desired characteristics is well within the skill of those skilled in the art, once armed with this disclosure.

In one embodiment of the method, the carrier is present in a range from about 5 percent to about 95 percent by weight of the composition. Preferably, the carrier is present in a range from about 20 percent to about 60 percent by weight of the composition.

In one embodiment of the method, the mixture further comprises an excipient. Preferably, the excipient is at least one of stearic acid, capric acid, or tricaprin, trilaurin, trimyristin, tripalmitin, tristearin, hydrogenated coco-glycerides, glyceryl monostearate, glyceryl behenate, glyceryl palmitostearate, lauric acid, palmitic acid, behenic acid, or cetyl palmitate. Preferably, the excipient is stearic acid. Preferably, the excipient is present in a range from about 1 percent to about 50 percent by weight of the composition. More preferably, the excipient is present in a range from about 1 percent to about 30 percent by weight of the composition.

In another embodiment of the present invention, a method of preparing a solid dispersion for delivering a beneficial agent with low water solubility to a patient is described, comprising melting the beneficial agent with a polymeric carrier; homogenizing the resulting mixture; cooling the mixture; and administering the dispersed beneficial agent to the patient.

Generally, beneficial agents may be administered to a patient by any known method in dosages ranging from about 0.01 to about 1.0 mmoles per kg body weight (and all combinations and subcombinations of dosage ranges and specific dosages therein). The useful dosage to be administered and the particular mode of administration will vary depending upon such factors as age, weight, and problem to be treated, as well as the particular beneficial agent used, as will be readily apparent to those skilled in the art. Typically, dosage is administered at lower levels and increased until the desirable diagnostic effect is achieved.

The method of this invention may be applied generally to commercially available gelatin capsules containing beneficial agent formulations. The invention has particular application to immediate-release gelatin encapsulated liquid, beneficial agent formulations that are conventionally manufactured and sold, but may be converted into controlled release dosage forms in accordance with this invention.

Melting the beneficial agent with the polymeric carrier is achieved by heating the mixture to a temperature greater than the melting point of the polymeric carrier, but lower than the temperature where the beneficial agent or polymeric carrier will degrade. Most carriers melt in a range from about −40° C. to about 60° C. No harm comes from heating the beneficial agent and carrier mixture to higher temperatures, provided that the temperature is lower than the temperature where the beneficial agent or polymeric carrier will degrade. Also, the beneficial agent need not be melted, as it will still solubilize in the melted carrier. Preferably, the mixture is heated in a range from about 60° C. to about 150° C. More preferably to about 135° C.

Homogenizing is achieved using conventional methods.

Cooling is achieved using conventional methods. Preferably, the mixture is rapidly cooled.

Beneficial agents used in the present invention include all those compounds known to have an effect on humans or animals that also have low water solubility. Such compounds include all those that can be categorized as Class 2 under the Biopharmaceutical Classification System (BCS) set out by the United States Food and Drug Administration (FDA). Determining which BCS Class a drug bellows in is a matter of routine experimentation, well known to those skilled in the art.

Exemplary beneficial agents that can be delivered by the osmotic system of this invention include prochlorperazine edisylate, ferrous sulfate, aminocaproic acid, potassium chloride, mecamylamine hydrochloride, procainamide hydrochloride, amphetamine sulfate, benzphetamine hydrochloride, isoproternol sulfate, methamphetamine hydrochloride, phenmetrazine hydrochloride, bethanechol chloride, metacholine chloride, pilocarpine hydrochloride, atropine sulfate, methascopolamine bromide, isopropamide iodide, tridihexethyl chloride, phenformin hydrochloride, methylphenidate hydrochloride, oxprenolol hydrochloride, metroprolol tartrate, cimetidine hydrochloride, diphenidol, meclizine hydrochloride, prochlorperazine maleate, phenoxybenzamine, thiethylperazine, maleate, anisindone, diphenadione erythrityl teranitrate, digoxin, isofurophate, reserpine, acetazolamide, methazolamide, bendroflumethiazide, chlorpropamide, tolazamide, chlormadinone acetate, phenaglycodol, allopurinol, aluminum aspirin, methotrexate, acetyl sulfisoxazole, erythromycin, progestins, estrogenic progrestational, corticosteroids, hydrocortisone, hydrocorticosterone acetate, cortisone acetate, triamcinolone, methyltesterone, 17 β-estradiol, ethinyl estradiol, ethinyl estradiol 3-methyl ether, prednisolone, 17-hydroxyprogesterone acetate, 19-nor-progesterone, norgestrel orethindone, norethiderone, progesterone, norgestrone, norethynodrel, aspirin, indomethacin, naproxen, fenoprofen, sulindac, diclofenac, indoprofen, nitroglycerin, propranolol, metroprolol, sodium valproate, valproic acid, taxanes such as paclitaxel, camptothecins such as 9-aminocamptothecin, oxprenolol, timolol, atenolol, alprenolol, cimetidine, clonidine, imipramine, levodopa, chloropropmazine, resperine, methyldopa, dihydroxyphenylalanine, pivaloyloxyethyl ester of a-methyldopa hydrochloride, theophylline, calcium gluconate ferrous lactate, ketoprofen, ibuprofen, cephalexin, haloperiodol, zomepirac, vincamine, diazepam, phenoxybenzamine, nifedipine, diltiazen, verapamil, lisinopril, captopril, ramipril, fosimopril, benazepril, libenzapril, cilazapril cilazaprilat, perindopril, zofenopril, enalapril, indalapril, qumapril, and the like. Other beneficial agents are known to the dispensing art as described in Pharmaceutical Sciences, by Remington, 14th Ed., 1979, published by Mack Publishing Co., Easton, Pa.; The Beneficial agent, The Nurse, The Patient, Including Current Beneficial agent Handbook, 1976, by Falconer et al., published by Saunder Company, Philadelphia, Pa.; Medical Chemistry, 3rd Ed., Vol. 1 and 2, by Burger, published by Wiley-Interscience, New York; and, Physician's Desk Reference, 55nd Ed., 1998, published by Medical Economics Co., New Jersey. The beneficial agent may be in various forms such as unchanged molecules, molecular complexes, pharmacologically acceptable salts such as hydrochloride, hydrobromide, sulfate, laurate, palmitate, phosphate, nitrite, nitrate, borate, acetate, maleate, tartrate, oleate, salicylate, and the like. For acidic beneficial agents, salts of metals, amines, or organic cations, for example quarternary ammonium can be used. Derivatives of beneficial agents, such as bases, ester, ether and amide can be used.

Beneficial agents having low water solubility, e.g., less than 50 micrograms/ml, are useful with the present invention. Beneficial agents include progesterone, megestrol acetate, topiramate, naproxen, flurbiprofen, ketoprofen, desipramine, diclofenac, itraconazole, piroxicam, carbamazepine, phenytoin, verapamil, indinavir sulfate, lamivudine, stavudine, nelfinavir mesylate, a combination of lamivudine and zidovudine, saquinavir mesylate, ritonavir, zidovudine, didanosine, nevirapine, ganciclovir, zalcitabine, fluoexetine hydrochloride, sertraline hydrochloride, paroxetine hydrochloride, bupropion hydrochloride, nefazodone hydrochloride, mirtazpine, auroix, mianserin hydrochloride, zanamivir, olanzapine, risperidone, quetiapine fumurate, buspirone hydrochloride, alprazolam, lorazepam, leotan, clorazepate dipotassium, clozapine, sulpiride, amisulpride, methylphenidate hydrochloride, and pemoline.

Preferably, the beneficial agents include progesterone, megestrol acetate, topiramate, naproxen, flurbiprofen, ketoprofen, desipramine, diclofenac, itraconazole, piroxicam, carbamazepine, phenytoin, and verapamil. More preferably, such compounds include progesterone, megestrol acetate, topiramate, and naproxen. Most preferably, the beneficial agent is progesterone.

In one embodiment of the method, the beneficial agent is present in a range from about 0.0001 percent to about 95 percent by weight of the composition. Preferably, the beneficial agent is present in a range from about 1 percent to about 20 percent by weight of the composition.

In one embodiment of the method, the carrier to beneficial agent ratio is about 10 to about 1.

In one embodiment of the method, the carrier to beneficial agent ratio is about 10 to about 5.

The carrier is a block copolymer of propylene oxide and ethylene oxide, a block copolymer derived from the addition of ethylene oxide and propylene oxide to ethylenediamine, polyethelene glycol, or polyethylene oxide.

In one embodiment of the method, the block copolymer of propylene oxide and ethylene oxide is of a formula HO-(ethylene oxide)ₓ-(propylene oxide)_{y}-(ethylene oxide)_{x′}-H. Preferably, x is in a range from about 2 to about 150, y is in a range from about 20 to about 70, and x′ is in a range from about 2 to about 150. Block copolymers of propylene oxide and ethylene oxide are available under the tradename PLURONIC from BASF Corporation, New Jersey, USA. PLURONIC block copolymers are pharmaceutical excipients listed in the US and British Pharmacopoeia.

Where relatively lower beneficial agent concentrations are desired, i.e., where the carrier to beneficial agent ratio is about 10 to about 1, preferably, x is in a range from about 20 to about 150, y is in a range from about 20 to about 70, and x′ is in a range from about 20 to about 150.

Where relatively higher beneficial agent concentrations are desired, i.e., where the carrier to beneficial agent ratio is about 10 to about 5, preferably, x is in a range from about 2 to about 80, y is in a range from about 20 to about 70, and x′ is in a range from about 2 to about 80.

In one embodiment of the method, x is about 41, y is about 16, and x′ is about 41. Such a block copolymer of propylene oxide and ethylene oxide is available under the tradename PLURONIC F38.

In one embodiment of the method, x is about 79, y is about 28, and x′ is about 79. Such a block copolymer of propylene oxide and ethylene oxide is available under the tradename PLURONIC F68.

In one embodiment of the method, x is about 64, y is about 37, and x′ is about 64. Such a block copolymer of propylene oxide and ethylene oxide is available under the tradename PLURONIC F87.

In one embodiment of the method, x is about 26, y is about 39, and x′ is about 26. Such a block copolymer of propylene oxide and ethylene oxide is available under the tradename PLURONIC F85.

In one embodiment of the method, x is about 141, y is about 44, and x′ is about 141. Such a block copolymer of propylene oxide and ethylene oxide is available under the tradename PLURONIC F108.

In one embodiment of the method, x is about 101, y is about 56, and x′ is about 101. Such a block copolymer of propylene oxide and ethylene oxide is available under the tradename PLURONIC F127.

Block copolymers derived from the addition of ethylene oxide and propylene oxide to ethylenediamine are available under the tradename TETRONIC from BASF Corporation, New Jersey, USA. The hydrophobic and hydrophilic parts of the molecule can be selectively varied to give a wide range of functional characteristics to give specific carrier requirements, and selection of the desired characteristics is well within the skill of those skilled in the art, once armed with this disclosure.

In one embodiment of the method, the carrier is present in a range from about 5 percent to about 95 percent by weight of the composition. Preferably, the carrier is present in a range from about 20 percent to about 60 percent by weight of the composition.

In one embodiment of the method, the mixture further comprises an excipient. Preferably, the excipient is at least one of stearic acid, capric acid, or tricaprin, trilaurin, trimyristin, tripalmitin, tristearin, hydrogenated coco-glycerides, glyceryl monostearate, glyceryl behenate, glyceryl palmitostearate, lauric acid, palmitic acid, behenic acid, or cetyl palmitate. Preferably, the excipient is stearic acid. Preferably, the excipient is present in a range from about 1 percent to about 50 percent by weight of the composition. More preferably, the excipient is present in a range from about 1 percent to about 30 percent by weight of the composition.

In terms of beneficial agent delivery systems, excellent results have been achieved with liquid beneficial agent formulations that allow a beneficial agent to be more readily absorbed through a patient's gastrointestinal membranes and into the bloodstream. For example, in the L-OROS™ HARDCAP™ beneficial agent delivery system, a beneficial agent layer and an osmotic engine are encased in a hard capsule surrounded by a rate-controlling semipermeable membrane, as described in U.S. Pat. Nos. 6,596,314, 6,419,952, and 6,174,547. The disclosures of each of the foregoing documents are hereby incorporated herein by reference in their entireties. In summary, a barrier layer, composed of an inert substance, separates the beneficial agent layer from the osmotic engine, preventing the beneficial agent from reacting with the osmotic engine. A delivery orifice, laser drilled in the membrane at the end opposite from the osmotic engine, provides an outlet for the beneficial agent.

Thus, in yet another embodiment of the present invention, a beneficial agent delivery system is described comprising a capsule having an orifice and surrounding an osmotic engine layer, a barrier layer, and a beneficial agent layer, wherein the beneficial agent layer comprises a beneficial agent dispersed in a polymeric carrier by mixing the beneficial agent with melted carrier.

The present invention is further described in the following examples.

### EXAMPLES

### Example 1

Progesterone is a naturally occurring steroid with low water solubility (12 μg/ml at 37° C. in artificial intestinal fluid (“AIF”). Progesterone is generally used for contraception (in formulations such as PROGESTASERT or THERAPIX), and is also prescribed to prevent spontaneous abortion or premature delivery. Its chemical name is pregn-4-ene-3,20-dione. It has an empirical formula of C₂₁H₃₀O₂ and a molecular weight of 314.5. The melting point of progesterone is 127-131° C.

The desired ratio, for example 10:1 or 10:5, of PLURONIC copolymer and progesterone were added to the bowl of a polymer mixer (30 cc or 10 cc). An oil heater (30 cc polymer mixer) or electrical heater (10 cc polymer mixer) was used to raise the temperature of the mixture to 135° C. The mixture melted and was stirred for 15 min at 108 rpm (30 cc polymer mixer) or 56 rpm (10 cc polymer mixer). The resulting homogeneous hot melt solutions were rapidly cooled with oil (30 cc polymer mixer) or chilling water (10 cc polymer mixer) to obtain solid dispersions.

### Example 2

The desired ratio of PLURONIC copolymer, progesterone, and excipient were added to the bowl of a polymer mixer (30 cc or 10 cc). An oil heater (30 cc polymer mixer) or electrical heater (10 cc polymer mixer) was used to raise the temperature of the mixture to 135° C. The mixture melted and was stirred for 15 min at 108 rpm (30 cc polymer mixer) or 56 rpm (10 cc polymer mixer). The resulting homogeneous hot melt solutions were rapidly cooled with oil (30 cc polymer mixer) or chilling water (10 cc polymer mixer) to obtain solid dispersions.

### Example 3

Samples as indicated below containing ˜4 mg of progesterone were added to 15 ml AIF and then were shaken in a water bath at 37° C. The particle size of the progesterone precipitation was measured with Horiba LA-910 laser scattering particle size analyzer.

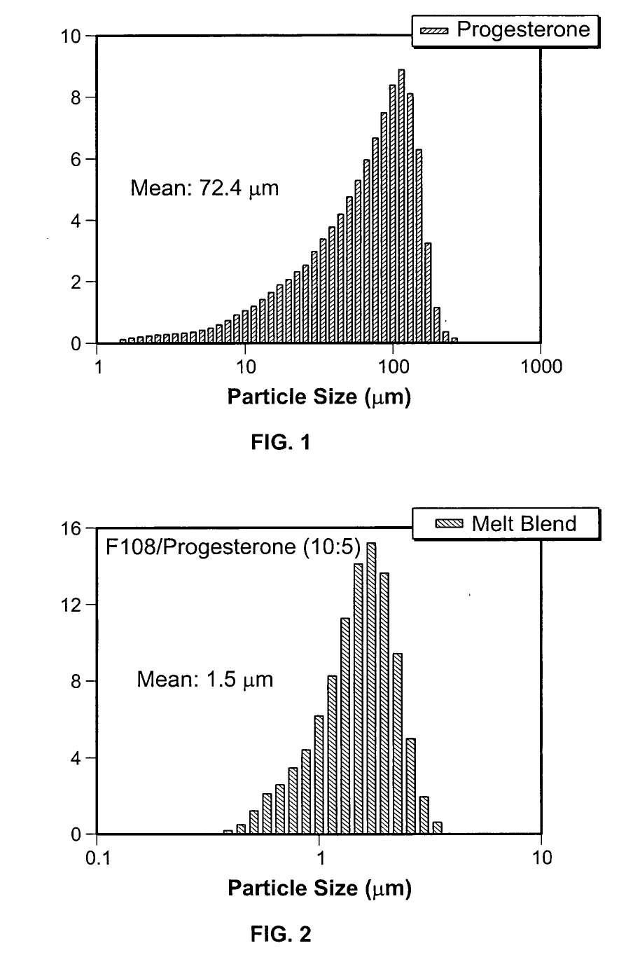

As shown in FIG. 1, the mean particle size of pure progesterone was 72.4 μm. FIG. 2 shows the particle size of progesterone precipitated from melt blend formulation PLURONIC F108 copolymer/progesterone (10:5) made according to the methods of Example 1. The particle size of the beneficial agent decreased from 72.4 μm in its pure form to 1.5 μm in the mixture made according to the methods of Example 1.

### Example 4

Samples as indicated below were mixed with AIF and shaken in a water bath at 37° C. for 6 h. The mixtures were filtered through a 0.45 μm cellulose nitrate membrane with vacuum filter. The particles were washed with deionized water several times to remove Pluronic®. Samples were dried in an oven at 30° C. overnight. Differential scanning calorimetry measurements were carried out using a TA Instruments 2920. Dry grade 5 nitrogen was used as purge gas. The samples were encapsulated in aluminum hermetically sealed sample pans. The thermal program applied to all samples equilibrated at 20° C. The sample was ramped 10° C. per minute to 150° C.

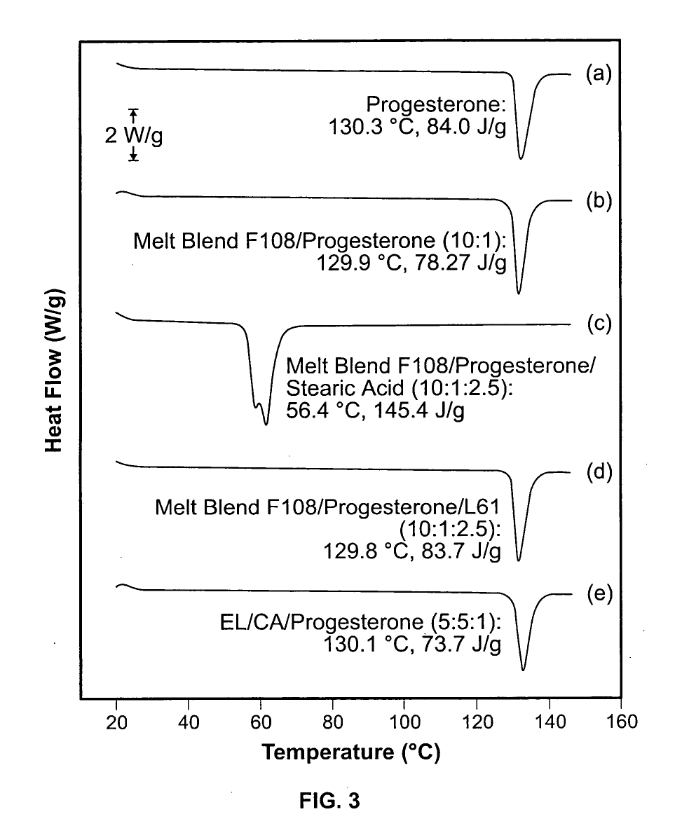

As shown in FIG. 3, differential scanning calorimetry (DSC) results are shown for samples including a) pure progesterone, b) PLURONIC copolymer F108/progesterone (10:1) made according to the methods of Example 1, c) PLURONIC copolymer F108/progestrone/stearic acid (10:1:2.5) made according to the methods of Example 2, d) PLURONIC copolymer F108/progesterone/L61 liquid PLURONIC copolymer having x=2, y=30, and x′=2 (10:1:2.5) made according to the methods of Example 1, and e) self-emulsifying formulation (SEF) CREMOPHOR EL brand polyethoxylated castor oil /capric acid (“CA”)/progesterone (5:5:1). Samples a, b, d and e had a sharp endothermic peak at ˜130° C. that corresponds to the melting point of progesterone, indicating PLURONIC copolymer and SEF did not change the crystallinity of the progesterone.

In contrast, sample c showed only one peak at 56.4° C. This complete absence of crystalline progesterone peak suggests that the melting point and the crystallinity of progesterone is reduced with the presence of stearic acid.

### Example 5

Dissolution studies were performed using the USP II systems. Samples as indicated below equivalent to 30 mg progesterone were added to 900 ml AIF (pH=6.8). The temperature of the dissolution medium was maintained at 37° C. The concentration of progesterone was measured with online UV at 249 nm.

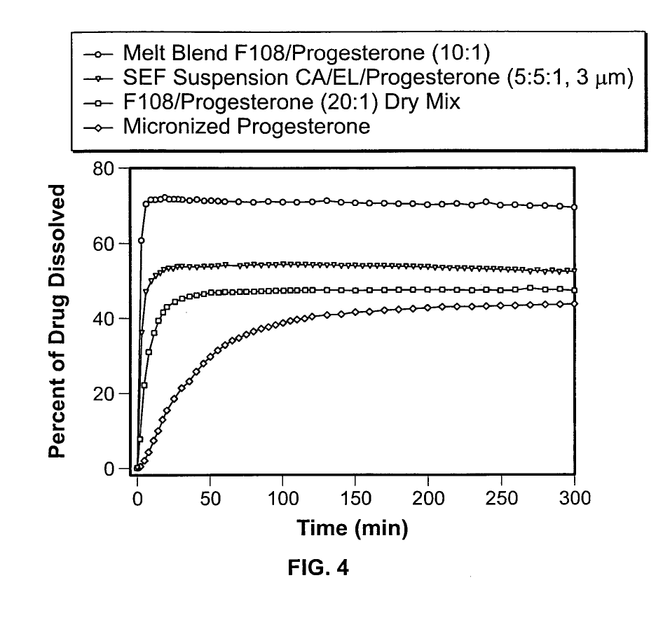

FIG. 4 shows the dissolution profiles of a) PLURONIC copolymer F108/progestrone (10:1) made according to the methods of Example 1; b) self-emulsifying formulation (SEF) EL/CA/progesterone (5:5:1); c) dry (i.e., not melted) mixture of PLURONIC copolymer F108/progestrone (20:1); and d) pure progesterone. Sample a had the best dissolution.

### Example 6

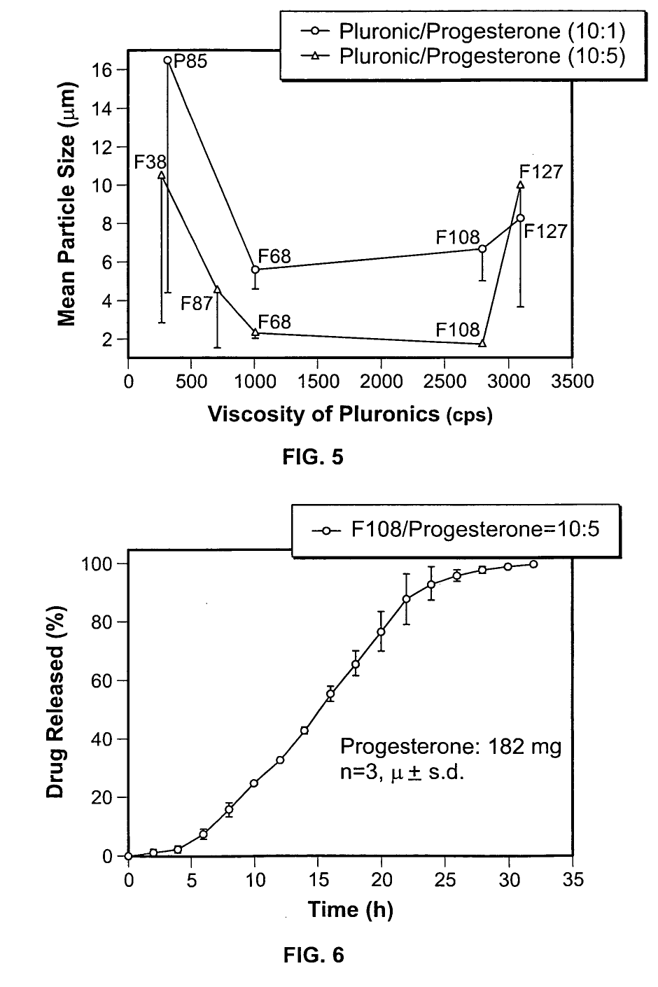

Samples were made according to Examples 1 and 2. FIG. 5 shows the particle size of precipitated progesterone from various melt blend formulations versus the viscosity of PLURONIC copolymer used. For both 10:1 and 10:5 beneficial agent loading systems, optimal viscosity exists for minimizing particle size.

Lower viscosity facilitates the dispersion of beneficial agent in polymer at elevated temperature, which can help reduce the particle size of beneficial agent in the polymer matrix. However, the growth rate of crystallization may be higher at lower viscosity system during the cooling process. The optimal carrier for low beneficial agent loading system is PLURONIC copolymer F68. Due to the much smaller molecules of progesterone compared with the PLURONIC copolymer molecules, increasing the beneficial agent loading may reduce the viscosity of the molten mixture, and the best formulation for higher beneficial agent loading system shifts to PLURONIC copolymer with higher viscosity. The optimal carrier for higher beneficial agent loading system is PLURONIC copolymer F108.

### Example 7

Samples were made according to Examples 1 and 2.

An L-OROS™ HARDCAP™ beneficial agent delivery system, as described in U.S. Pat. Nos. 6,596,314, 6,419,952, and 6,174,547 was prepared for dispensing progesterone in a controlled manner over a prolonged period of time.

A bilayer tablet with an osmotic engine layer (350 mg), comprising 63.67% POLYOX 303 polyethylene oxide, 30% NaCl, 5% HPMC E5 hydroxypropyl methylcellulose, 1% red ferric oxide, 0.25% magnesium stearate and 0.08% butylated hydroxytoluene (“BHT”) by weight, and a barrier layer, comprising 100 mg KOLLIDON SR polyvinyl acetate and polyvinylpyrrolidone blend with minor amounts of sodium lauryl sulfate and colloidal silica sustained release excipient available from BASF Corporation, was inserted into the hydroxypropyl methylcellulose (“HPMC”) “0” size capsule (sub-coated with 9.5 mg SURELEASE aqueous ethyl cellulose dispersion) with the barrier side first.

The capsule assembly was coated then with a 100 mg semipermeable membrane comprising 90% cellulose acetate 398-10 and 10% PLURONIC copolymer F68 by weight. An orifice with diameter of 0.0625 inch was drilled through the module.

PLURONIC copolymer and progesterone (10:5 by weight) were added to the bowl of a polymer mixer (10 cc). An electrical heater was used to raise the temperature of the mixture to 135° C. The mixture melted and was stirred for 15 min at 56 rpm. The resulting homogeneous hot melt solution was rapidly filled into the module through the orifice with 1 ml syringe. This hot melt formulation was cooled down, and it solidified at ambient conditions. An average of 546 mg melt blend formulation was filled into the module, containing 182 mg progesterone.

The in vitro release rate of progesterone was measured at 37° C. in AIF (pH=6.8). Samples were analyzed by UV spectroscopy at 245 nm. FIG. 6 shows a zero order release profile for the progesterone for ˜20 h.

The disclosures of each patent, patent application, and publication cited or described in this document are hereby incorporated herein by reference, in their entireties.

Each recited range includes all combinations and subcombinations of ranges, as well as specific numerals contained therein.

Various modifications of the invention, in addition to those described herein, will be apparent to those skilled in the art from the foregoing description. Such modifications are also intended to fall within the scope of the appended claims.

1. A method of preparing a solid dispersion for delivering a beneficial agent with low water solubility, comprising:
melting the beneficial agent with a polymeric carrier;
homogenizing the resulting mixture; and
cooling the mixture.

2. The method of claim 1 wherein the mixture is heated to a temperature greater than the melting point of the polymeric carrier, and lower than the temperature where the beneficial agent or polymeric carrier would degrade.

3. The method of claim 1 wherein the mixture is heated in a range from about 60° C. to about 150° C.

4. The method of claim 1 wherein the mixture is rapidly cooled.

5. The method of claim 1 wherein the beneficial agent is selected from at least one of the Class II pharmaceuticals.

6. The method of claim 1 wherein the beneficial agent is selected from at least one of progesterone, megestrol acetate, topiramate, naproxen, flurbiprofen, ketoprofen, desipramine, diclofenac, itraconazole, piroxicam, carbamazepine, phenytoin, and verapamil.

7. The method of claim 1 wherein the beneficial agent is selected from at least one of progesterone, megestrol acetate, topiramate, and naproxen.

8. The method of claim 1 wherein the beneficial agent is progesterone.

9. The method of claim 1 wherein the beneficial agent is present in a range from about 0.0001 percent to about 95 percent by weight of the composition.

10. The method of claim 1 wherein the beneficial agent is present in a range from about 1 percent to about 20 percent by weight of the composition.

11. The method of claim 1 wherein the carrier to beneficial agent ratio is about 10 to about 1.

12. The method of claim 1 wherein the carrier to beneficial agent ratio is about 10 to about 5.

13. The method of claim 1 wherein the carrier is a block copolymer of propylene oxide and ethylene oxide, a block copolymers derived from the addition of ethylene oxide and propylene oxide to ethylenediamine, polyethelene glycol, or polyethylene oxide.

14. The method of claim 13 wherein the copolymer is of a formula HO-(ethylene oxide)ₓ-(propylene oxide)_{y}-(ethylene oxide)_{x′}-H.

15. The method of claim 14 wherein x is in a range from about 2 to about 150, y is in a range from about 20 to about 70, and x′ is in a range from about 2 to about 150.

16. The method of claim 14 wherein x is in a range from about 20 to about 150, y is in a range from about 20 to about 70, and x′ is in a range from about 20 to about 150.

17. The method of claim 14 wherein x is in a range from about 2 to about 80, y is in a range from about 20 to about 70, and x′ is in a range from about 2 to about 80.

18. The method of claim 14 wherein x is about 41, y is about 16, and x′ is about 41.

19. The method of claim 14 wherein x is about 79, y is about 28, and x′ is about 79.

20. The method of claim 11 wherein the carrier is a block copolymer of propylene oxide and ethylene oxide, wherein the copolymer is of a formula HO-(ethylene oxide)ₓ-(propylene oxide)_{y}-(ethylene oxide)_{x′}-H, and wherein x is about 79, y is about 28, and x′ is about 79.

21. The method of claim 14 wherein x is about 64, y is about 37, and x′ is about 64.

22. The method of claim 14 wherein x is about 26, y is about 39, and x′ is about 26.

23. The method of claim 14 wherein x is about 141, y is about 44, and x′ is about 141.

24. The method of claim 12 wherein the carrier is a block copolymer of propylene oxide and ethylene oxide, wherein the copolymer is of a formula HO-(ethylene oxide)ₓ-(propylene oxide)_{y}-(ethylene oxide)_{x′}-H, and wherein x is about 141, y is about 44, and x′ is about 141.

25. The method of claim 14 wherein x is about 101, y is about 56, and x′ is about 101.

26. The method of claim 1 wherein the carrier is present in a range from about 5 percent to about 95 percent by weight of the composition.

27. The method of claim 1 wherein the carrier is present in a range from about 20 percent to about 60 percent by weight of the composition.

28. The method of claim 1 wherein the mixture further comprises an excipient.

29. The method of claim 28 wherein the excipient is at least one of stearic acid, capric acid, or tricaprin, trilaurin, trimyristin, tripalmitin, tristearin, hydrogenated coco-glycerides, glyceryl monostearate, glyceryl behenate, glyceryl palmitostearate, lauric acid, palmitic acid, behenic acid, or cetyl palmitate.

30. The method of claim 28 wherein the excipient is stearic acid.

31. The method of claim 28 wherein the excipient is present in a range from about 1 percent to about 50 percent by weight of the composition.

32. The method of claim 28 wherein the excipient is present in a range from about 1 percent to about 30 percent by weight of the composition.

33. A method of preparing a solid dispersion for delivering a beneficial agent with low water solubility to a patient, comprising:
melting the beneficial agent with a polymeric carrier;
homogenizing the resulting mixture;
cooling the mixture; and
administering the dispersed beneficial agent to the patient.

34. The method of claim 33 wherein the mixture is heated to a temperature greater than the melting point of the polymeric carrier, and lower than the temperature where the beneficial agent or polymeric carrier would degrade.

35. The method of claim 33 wherein the mixture is heated in a range from about 60° C. to about 150° C.

36. The method of claim 33 wherein the mixture is rapidly cooled.

37. The method of claim 33 wherein the beneficial agent is selected from at least one of the Class II pharmaceuticals.

38. The method of claim 33 wherein the beneficial agent is selected from at least one of progesterone, megestrol acetate, topiramate, naproxen, flurbiprofen, ketoprofen, desipramine, diclofenac, itraconazole, piroxicam, carbamazepine, phenytoin, and verapamil.

39. The method of claim 33 wherein the beneficial agent is selected from at least one of progesterone, megestrol acetate, topiramate, and naproxen.

40. The method of claim 33 wherein the beneficial agent is progesterone.

41. The method of claim 33 wherein the beneficial agent is present in a range from about 0.0001 percent to about 95 percent by weight of the composition.

42. The method of claim 33 wherein the beneficial agent is present in a range from about 1 percent to about 20 percent by weight of the composition.

43. The method of claim 33 wherein the carrier to beneficial agent ratio is about 10 to about 1.

44. The method of claim 33 wherein the carrier to beneficial agent ratio is about 10 to about 5.

45. The method of claim 33 wherein the carrier is a block copolymer of propylene oxide and ethylene oxide, a block copolymers derived from the addition of ethylene oxide and propylene oxide to ethylenediamine, polyethelene glycol, or polyethylene oxide.

46. The method of claim 45 wherein the copolymer is of a formula HO-(ethylene oxide)ₓ-(propylene oxide)_{y}-(ethylene oxide)_{x′}-H.

47. The method of claim 46 wherein x is in a range from about 2 to about 150, y is in a range from about 20 to about 70, and x′ is in a range from about 2 to about 150.

48. The method of claim 46 wherein x is in a range from about 20 to about 150, y is in a range from about 20 to about 70, and x′ is in a range from about 20 to about 150.

49. The method of claim 46 wherein x is in a range from about 2 to about 80, y is in a range from about 20 to about 70, and x′ is in a range from about 2 to about 80

50. The method of claim 46 wherein x is about 41, y is about 16, and x′ is about 41.

51. The method of claim 46 wherein x is about 79, y is about 28, and x′ is about 79.

52. The method of claim 43 wherein the carrier is a block copolymer of propylene oxide and ethylene oxide, wherein the copolymer is of a formula HO-(ethylene oxide)ₓ-(propylene oxide)_{y}-(ethylene oxide)_{x′}-H, and wherein x is about 79, y is about 28, and x′ is about 79.

53. The method of claim 46 wherein x is about 64, y is about 37, and x′ is about 64.

54. The method of claim 46 wherein x is about 26, y is about 39, and x′ is about 26.

55. The method of claim 46 wherein x is about 141, y is about 44, and x′ is about 141.

56. The method of claim 44 wherein the carrier is a block copolymer of propylene oxide and ethylene oxide, wherein the copolymer is of a formula HO-(ethylene oxide)ₓ-(propylene oxide)_{y}-(ethylene oxide)_{x′}-H, and wherein x is about 141, y is about 44, and x′ is about 141.

57. The method of claim 46 wherein x is about 101, y is about 56, and x′ is about 101.

58. The method of claim 33 wherein the carrier is present in a range from about 5 percent to about 95 percent by weight of the composition.

59. The method of claim 33 wherein the carrier is present in a range from about 20 percent to about 60 percent by weight of the composition.

60. The method of claim 33 wherein the mixture further comprises an excipient.

61. The method of claim 60 wherein the excipient is at least one of stearic acid, capric acid, or tricaprin, trilaurin, trimyristin, tripalmitin, tristearin, hydrogenated coco-glycerides, glyceryl monostearate, glyceryl behenate, glyceryl palmitostearate, lauric acid, palmitic acid, behenic acid, or cetyl palmitate.

62. The method of claim 60 wherein the excipient is stearic acid.

63. The method of claim 60 wherein the excipient is present in a range from about 1 percent to about 50 percent by weight of the composition.

64. The method of claim 60 wherein the excipient is present in a range from about 1 percent to about 30 percent by weight of the composition.

65. A beneficial agent delivery system comprising:
a capsule having an orifice and surrounding an osmotic engine layer, a barrier layer, and a beneficial agent layer, wherein the beneficial agent layer comprises a beneficial agent dispersed in a polymeric carrier by mixing the beneficial agent with melted carrier.
